(19) United States
(12) Patent Application Publication
(10) Pub. No.: US 2021/0145783 A1
(43) Pub. Date: May 20, 2021

(54) Compositions and Methods for Treating Sickle Cell Disease

(71) Applicants: Augusta University Research Institute, Inc., Augusta, GA (US); Bar Ilan University, Ramat-Gan (IL); Ramot at Tel-Aviv University Ltd., Tel Aviv (IL)

(72) Inventors: Betty S. PACE, Augusta, GA (US); Abraham Nudelman, Rehovot (IL); Zvi MALIK, Kfar Haroeh (IL); Ada REPHAELI, Tel Aviv (IL)

(21) Appl. No.: 17/099,455

(22) Filed: Nov. 16, 2020

Related U.S. Application Data
(60) Provisional application No. 62/935,302, filed on Nov. 14, 2019.

(51) Int. Cl. A61K 31/22; A61K 31/17; A61K 9/00; A61P 7/06

(52) U.S. Cl. CPC A61K 31/22; A61K 31/17; A61P 7/06; A61K 9/0048; A61K 9/0053

(57) ABSTRACT

It has been found that the prodrug 1-(butyryloxy)ethyl-5-amino-4-oxopentanoate (AN-233), an oral active conjugate of BA (histone deacetylase inhibitor) and ALA (heme precursor), is useful for the treatment of hemoglobinopathies including but not limited to sickle cell disease and thalassemias. In one embodiment, AN-233 activates γ-globin transcription, induces HbF expression, produces an anti-sickling effect, or combinations thereof when administered to a subject in need thereof.

### CROSS-REFERENCE TO RELATED APPLICATIONS

This application claims benefit of and priority to U.S. Patent Application No. 62/935,302 filed on Nov. 14, 2019, which is incorporated by reference in its entirety.

### STATEMENT REGARDING FEDERALLY SPONSORED RESEARCH

This invention was made with government support under HL069234 awarded by the National Institutes of Health. The government has certain rights in the invention

### TECHNICAL FIELD OF THE INVENTION

Aspects of the invention are generally directed to compounds and methods for treating sickle cell disease.

### BACKGROUND OF THE INVENTION

Sickle cell disease (SCD) is a group of hematologic disorders that arise from mutations in the structural gene encoding a subunit of hemoglobin (Hb). A single point mutation, A to T at the sixth codon of the β-globin gene results in the production of HbS (Pauling, L., et al., Science, 110:543-548 (1949)). The homozygous form of the mutation produces sickle cell anemia, in which HbS polymerizes under deoxygenated conditions leading to formation of sickle-shaped red blood cells (RBCs). In the United States, approximately 100,000 individuals are affected with SCD and worldwide over 330,000 babies are born annually (Modell, B., et al., Bull. World Health Organ., 86:480-487(2008)). The different forms of SCD are characterized by chronic hemolysis, anemia and impair blood flow by sickle RBCs, leading to recurrent painful vaso-occlusive episodes and other complications such as infection, acute chest, splenic sequestration, and end organ damage (Piel, F. B., et al., N. Engl. J. Med., 376:561-1573 (2017); Sundd, P., et al., Annu. Rev. Pathol., 14 263-292 (2019)).

The development of pharmacologic agents that induce HbF expression is an effective strategy for treating people with SCD because HbF exerts anti-sickling effects through formation of HbS/HbF hybrid molecules (Poillon, W. N., et al., Proc. Natl. Acad. Sci. U.S.A, 90:5039-5043(1993)). Studies from the Comprehensive Study of Sickle Cell Disease demonstrated higher HbF levels improve long-term survival of persons with sickle cell anemia (Platt, O. S., et al., N. Engl. J. Med., 330:1639-1644 (1994)). After demonstrated efficacy in the Multicenter Hydroxyurea Study, in 1998 hydroxyurea (HU) became the only Food and Drug Administration-approved drug proven to induce HbF in SCD patients. The use of HU is limited by a significant non-responder rate, the need for close monitoring of blood counts for bone marrow toxicity, infertility, and patient concerns with taking a chemotherapy class agent (Charache, S., et al., N. Engl. J. Med., 332:1317-1322 (1995); Sahoo, K., et al., J. Assoc. Physicians India, 65:22-25 (2017)).

Several groups have previously reported robust HbF induction in SCD patients by the histone deacetylase inhibitor butyric acid (BA), however rapid metabolism of BA when given by oral administration hindered clinical development. Subsequent studies demonstrated the ability of BA to activate p38 MAPK signaling and CREB1 phosphorylation to achieve γ-globin gene trans-activation [14]. Individuals treated with intravenous BA showed robust HbF induction, but continuous treatment produced anti-proliferative effects in the bone marrow, requiring intermitted drug dosing. Clinical trials using the oral analogue dimethylbuty-rate induced HbF in β-thalassemia patients, but proved less effective in SCD.

Therefore, there is a need for the development of additional less toxic and effective therapies.

It is an object of the invention to provide new compositions and methods of their use to treat sickle cell disease.

It is another object of the invention to provide compositions and methods for treating subjects with one or more mutations in the beta-globin gene (HBB), or an expression control sequence thereof.

It is another object of the invention to provide compositions and methods for treating subjects with sickle cell disease, beta thalassemia, or variants or related diseases or conditions thereof.

It is another object of the invention to provide compositions and methods for reducing one or more symptoms of sickle cell disease, beta thalassemia, or variants or related diseases or conditions thereof.

It is a further object of the invention to provide treatments for sickle cell disease with fewer, or less severe side effects, greater efficacy, greater response rate, or combinations thereof compared to existing therapies such as hydroxyurea.

### SUMMARY OF THE INVENTION

It has been found that the prodrug 1-(butyryloxy)ethyl-5-amino-4-oxopentanoate (AN-233), an oral active conjugate of BA (histone deacetylase inhibitor) and ALA (heme precursor), is useful for the treatment of hemoglobinopathies including but not limited to SCD and thalassemias. In one embodiment, AN-233 activates γ-globin transcription, induces HbF expression, produces an anti-sickling effect, or combinations thereof.

One embodiment provides methods of treating sickle cell disease (SCD) or complications of SCD by administering an effective amount of AN-233 or pharmacologically active salts, derivatives, or analogues thereof to induce or increase expression of fetal hemoglobin (HbF) in a subject in need thereof. Another embodiment provides a method for treating SCD or complications related to SCD by administering AN-233 in combination or alternation with hydroxyurea (HU). In one aspect, the subject treated with the combination of AN-233 and HU is typically unresponsive or does not respond well to HU treatment alone. In some embodiments subjects for treatment with the combination of AN-233 and HU have reduced expression of OCTN1 relative to subjects that respond well to HU treatment alone.

One embodiment provides methods for treating retinopathy due to SCD by administering AN-233 optionally in combination or alternation with HU in an amount effective to increase HbF expression in retinal pigment epithelial cells. In some embodiments, AN-233 is administered to the eye, for example intravitreally.

In one embodiment AN-233 optionally in combination or alternation with HU is administered in an effective amount to increase HbF expression in a subject in need thereof to reduce one or more symptoms of a sickle cell disorder in the subject. The sickle cell disorder can be a sickle cell disease such as sickle cell anemia. Typically, the subject has at least one allele of sickle cell hemoglobin (HbS). In some embodiments, the subject has one allele of HbS and one allele of hemoglobin C (HbC), one allele of hemoglobin E (HbE), one allele of β−0 thalassemia, or one allele of β+thalassemia. In some embodiments, the subject has two alleles of HbS.

AN-233 alone or in combination or alternation with HU can be used in combination or alternation with another therapeutic agents to treat SCD or complications of SCD. Representative additional therapeutic agents include, but are not limited to L-glutamine oral powder, crizanlizumab, Voxelotor, fumaric esters, pain relieving drugs, and combinations thereof. In one embodiment the combination of AN-233 with HU, and optionally with additional therapeutic agent can be formulated in a unit dose form. One embodiment provides a pharmaceutical composition containing AN-233 and HU, optionally including an excipient. An exemplary complication of SCD that can be treated with the disclosed compositions includes but is not limited to retinal complications.

In another embodiment AN-233 optionally in combination or alternation with HU is administered to a subject in need thereof in an effective amount to increase HbF expression to reduce one or more symptoms of a beta-thalassemia in the subject. The beta-thalassemia can be, for example, thalassemia minor, thalassemia intermedia, and thalassemia major.

In some embodiments, AN-233 optionally in combination or alternation with HU is administered to a subject in need thereof in an effective amount to increase HbF expression to compensate for a mutation in the human beta-globin gene. Compensating for a mutation in the human beta globin gene includes inducing expression of HbF.

Other embodiments provide methods of increasing HbF expression in hemoglobin synthesizing cells. The methods typically include contacting cells with an effective amount of a AN-233 optionally in combination or alternation with HU to increase HbF expression in the cells. In some embodiments the cells are erythroid precursor cells. In other embodiments, the cells are non-erythriod cells such as macrophage, retinal pigment cells, or alveolar epithelial cells.

One embodiment provides a pharmaceutical composition containing an effective amount of AN-233 optionally in combination with HU to increase HbF expression in a subject in need thereof. In some embodiments the dosage is 1 mg/kg to about 50 mg/kg. In other embodiments the dosage is 0.1 g and 2.0 g per day. AN-233 optionally in combination or alternation with HU can be administered as part of a dosage regime. The dosage regime can include dose escalation.

In some embodiments the dosing of hydroxyurea for sickle cell disease calls for the administration of an initial dose of 15 mg/kg/day in the form of a single dose, with monitoring of the patient's blood count every 2 weeks. If the blood counts are in an acceptable range, the dose may be increased by 5 mg/kg/day every 12 weeks until the MTD of 35 mg/kg/day is reached. Pharmaceutical compositions can contain 1 mg/kg to 50 mg/kg of AN-233 in combination with 1 mg/kg to 35 mg/kg of HU.

An exemplary dosage regime for treatment of a sickle cell disorder includes administering to a subject with a sickle cell disorder a low dose of AN-233 and administering to the subject escalating doses of the AN-233 until the dose is effective to reduce one or more symptoms of the sickle cell disorder.

Some of the disclosed methods include administering to the subject a second active agent, for example, vitamin supplements, nutritional supplements, anti-anxiety medication, anti-depression medication, anti-coagulants, clotting factors, anti-inflammatories, steroids such as corticosteroids, analgesic, etc. In some embodiments, the compositions are co-administered in combination with one or more additional active agents for treatment of sickle cell disease, beta-thalassemia, or a related disorder. Such additional active agents may include, but are not limited to, folic acid, penicillin or another antibiotics, preferably a quinolone or macrolide, antivirals, anti-malarial prophylactics, and analgesics to control pain. In some embodiments, the compositions are co-administered with one or more additional agents that increase expression of HbF, for example, hydroxyurea or fumaric acid esters.

Methods of selecting a subject with a mutation in a beta-globin gene for treatment are also disclosed. The methods typically include genotyping the beta-globin gene and expression control sequence thereof in DNA isolated from a biological sample obtained from the subject; determining if the beta-globin gene or expression control sequence includes a mutation; selecting the subject for treatment if the beta-globin gene or expression control sequence includes a mutation; and treating the subject with an effective amount of AN-233.

Still another method of treatment provides administering AN-233 in combination or alternation with HU to SCD subjects that are unresponsive to HU treatment alone. For example, m AN-233 can be administered to enhance the uptake of HU in subjects that are typically unresponsive to HU. Unresponsive to HU treatment means that the subject having SCD does not experience a significant therapeutic effect for treating their SCD from HU treatment. The increase in uptake of HU can also be accompanied by an increase in HbF expression.

### BRIEF DESCRIPTION OF THE DRAWINGS

FIGS. 1A-1D show that AN-233 increased γ-globin transcription and HbF synthesis. K562 cells were treated with AN-233 and ethanol (EtOH) vehicle control for 48 h. Total RNA and whole cell lysates were isolated for RT-qPCR and Western blot analysis. After treatments, K562 cells were fixed and stained for flow cytometry. All data are shown as the mean±SEM (N=5) and *p<0.05 was considered statistically significant; **p<0.01. FIG. 1A is a bar graph showing mRNA data generated by RT-qPCR under the different treatment conditions. In FIGS. 1B and 1C K562 cells were stained with fluorescein isothiocyanate (FITC) conjugated anti-HbF antibody and analyzed by flow cytometry. FIG. 1B shows representative histograms of cell populations that stained positive for HbF (F-cells) and FIG. 1C shows the quantitiative data in a bar graph. FIG. 1D shows Western blot analysis determined HbF levels with tubulin as internal loading control including a representative blot and quantitative data generated by densitometry analysis. UT=unteated, BA=butyric acid, HU=hydroxyurea; ALA=S-aminolevulinate, HEM=hemin; AN-233=1-(butyryloxy)ethyl-5-amino-4-oxopentanoate.

FIGS. 2A-2D show that AN-233 mediates heme biosynthesis and modulates several targets in vitro. K562 cells were induced with the prodrug AN-233 for 48 h and then analyzed for heme levels and HRI, eIF2α, and p38 MAPK protein levels. All data are shown as the mean±SEM (N=3 per group) and *p<0.05 was considered statistically significant. FIG. 1A is a bar graph showing intracellular heme levels measured using a colorimetric QuantiChrom™ Heme Assay Kit for K562 cells under the different treatment conditions; heme values were normalized by total protein. FIG. 2B shows Western blot analysis of phosphorylated HRI (p-HRI) and total HRI (t-HRI) levels. FIG. 2C shows Western blot analysis of phosphorylated eukaryotic translation initiation factor (eIF2αP) levels. FIG. 2D shows a representative gel and quantitative data for Western blot of p38 MAPK expression after AN-233 treatment.

FIGS. 3A-3E show that AN-233 induces HbF without changing HbS levels in sickle erythroid precursors. A second lot of AN-233 dissolved in water vehicle were used to treat primary sickle erythroid progenitors on day 8 for 48 h and used for the various studies. All data are shown as the mean±SEM (N=3 per group) and *p<0.05 was considered statistically significant. FIG. 3A shows representative histograms from flow cytometry analysis of sickle erythroid precursors stained with FITC-HbF antibody. In FIG. 3B F-cell levels were determined by flow cytometry and quantitative data generated by FlowJo™ analysis. In FIG. 3C the level of HbF protein was quantified by MFI generated by flow cytometry. In FIG. 3D Total protein lysates were isolated and used for Western blot for HbF and HbS protein; β-actin was the loading control. FIG. 3D provides a representative blot and the quantitative data generated by densitometry. In FIG. 3E erythroid precursors were treated with the various drugs 7o determine if AN-233 mediates anti-sickling effects, and then incubated in 2% oxygen overnight, fixed in 2% formaldehyde and the number of sickle-shaped erythroid progenitors counted by light microscopy. FIG. 3E provides images of sickle precursors for different conditions and summary of quantitative data for 1000 cells per triplicate for N=3 donors.

FIGS. 4A-4D show that AN-233 enhances histone acetylation in the γ-globin gene promoter. In FIG. 4A nuclear protein lysates were isolated from sickle erythroid precursors and used for Western blot analysis of acetylated histone H3 (AcH3) levels (N=3). Sickle erythroid progenitors treated under the different conditions=were used for ChP, Shown is AcH3 and AcH4 levels in the (4B) proximal γ-globin gene promoter, (4C) locus control region DNAse I hypersensitivity site 2 (LCR-HS2), and (4D) β-globin gene promoter; *p<0.05 was considered statistically significant.

FIGS. 5A-5D show that AN-233 increases HbF expression in β-YAC transgenic mice. In FIG. 5A 5-6 months old β-YAC transgenic mice were treated with 200 or 300 mg/kg of AN-233 dissolved in water for 4 weeks by intraperitoneal injections; water (vehicle) and hydroxyurea (HU) treatments were completed as controls to assess the in vivo effect of AN-233 (N=5 per group; 3 males and 2 females). In FIG. 5B blood samples were collected at week 0, 2 and 4 were stained with acridine orange for reticulocyte percent by flow cytometry. Data are shown as the mean±SEM and p<0.05 was considered significant; exact p-values are shown. In FIG. 5C peripheral blood was stained with FITC-conjugated anti-HbF antibody and flow cytometry performed to quantify the F-cells by FlowJo™ data analysis. In FIG. 5D the level of HbF expression was measured by MFI data generated by flow cytometry analysis.

### DETAILED DESCRIPTION OF THE INVENTION

### I. Definitions

The term “expression control sequence” refers to a nucleic acid sequence that controls and regulates the transcription and/or translation of another nucleic acid sequence. Control sequences that are suitable for prokaryotes, for example, include a promoter, optionally an operator sequence, a ribosome binding site, and the like. Eukaryotic cells are known to utilize promoters, polyadenylation signals, and enhancers.

The term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject. The test subject can be a subject afflicted with a genetic mutation in the beta-globin gene or an expression control sequence thereof, or a subject with a sickle cell disorder, a globinopathy, or a beta-thalassemia.

As used herein, the term “treating” includes alleviating the symptoms associated with a specific disorder or condition and/or preventing or eliminating said symptoms, including hemoglobinopathies.

### II. Methods of Treating Sickle Cell Disease, Beta-Thalassemias, and Related Disorders

A. Treatment of SCD with AN-233

Methods of increasing expression of HbF in cells by contacting the cells, for example erythroid and RPE cells, with an effective amount of AN-233, or pharmacologically active salt, derivative, analogue or prodrug thereof are disclosed. The methods can be used to compensate for a mutation in the human beta-globin gene in cells that have one or more mutations in the beta-globin gene or an expression control sequence thereof, for example mutations that result in the expression of the HbS form of hemaglobin. Compensating for the mutation includes but is not limited to increasing the amount of HbF and optionally reducing the amount of HbS in the subject compared to untreated subjects. The methods can be used for treating sickle cell disease, for example sickle cell anemia, and other hemoglobinopathies or thalassemias as well as complications related to SCD, for example retinopathy.

B. Treatment with AN-233 in Combination or Alternation with HU.

Some embodiments provide methods for treating SCD or complications thereof include administering AN-233 in combination or alternation with HU in amounts effective to induce or increase expression of HbF and optionally increase expression of OCTN1 in erythroid and retinal cells. It has been discovered that AN-233 induces the expression of HbF. In some embodiments, AN-233 can reduce the dosing of HU in SCD patients without compromising its therapeutic efficacy, and reduce or mitigate the toxic side effects associated with HU therapy.

Subjects with SCD that are unresponsive to HU treatment can be treated by administering a AN-233 in combination or alternation with HU. While AN-233 adjuvant therapy along with HU would certainly benefit SCD patients who respond to HU, it also has potential to work on those who do not respond to HU. In one embodiment, the “non-responders” express lower levels of OCTN1 than “responders.” The decreased expression of the transporter would result in decreased entry of HU into its target cells (erythroid progenitors) and thus decrease its pharmacological effect.

Reactivation of HbF, which is typically absent or expressed only at low levels in humans over six months of age, is considered a viable approach for treating children and adults with sickle cell disease, and other hemoglobinopathies and thalassemias. The methods disclosed herein typically include administering a AN-233, or pharmacologically active salt, derivative, analogue or prodrug thereof to a subject in need thereof to increase expression of HbF in the subject, to increase expression of OCTN1, or both.

C. Diseases to be Treated

The disclosed AN-233 compositions can be used to treat subjects with one or more mutations in the beta-globin gene (HBB gene). Mutations in the beta globin gene can cause sickle cell disease, beta thalassemia, or related diseases or conditions thereof. As discussed in more detail below, mutations in the beta-globin gene can be identified before or after manifestations of a disease's clinical symptoms. The compositions can be administered to a subject with one or more mutations in the beta-globin gene before or after the onset of clinical symptoms. Therefore, in some embodiments, the compositions are administered to a subject that has been diagnosed with one or more mutations in the beta-globin gene, but does not yet exhibit clinical symptoms. In some embodiments, the compositions are administered to a subject that is exhibiting one or more symptoms of a disease, condition, or syndrome associated with, or caused by one or more mutations in the beta-globin gene.

1. Sickle Cell Disease

Sickle cell disease (SCD) typically arises from a mutation substituting thymine for adenine in the sixth codon of the beta-chain gene of hemoglobin (i.e., GAG to GTG of the HBB gene). This mutation causes glutamate to valine substitution in position 6 of the Hb beta chain. The resulting Hb, referred to as HbS, has the physical properties of forming polymers under deoxy conditions. SCD is typically an autosomal recessive disorder. Therefore, in some embodiments, the disclosed compositions and methods are used to treated a subject homozygous for an autosomal recessive mutation in beta-chain gene of hemoglobin (i.e., homozygous for sickle cell hemoglobin (HbS)). Also referred to as HbSS disease or sickle cell anemia (the most common form), subjects homozygote for the S globin typically exhibit a severe or moderately severe phenotype and have the shortest survival of the hemaglobinopathies.

Sickle cell trait or the carrier state is the heterozygous form characterized by the presence of around 40% HbS, absence of anemia, inability to concentrate urine (isosthenuria), and hematuria. Under conditions leading to hypoxia, it may become a pathologic risk factor. Accordingly, in some embodiments, the disclosed compositions and methods are used to treat a subject heterozygous for an autosomal recessive mutation in the beta-chain gene of hemoglobin (i.e., heterozygous for HbS).

2. Beta-Thalassemia

One embodiment provides a method for treating thalassemia, for example β-thalassemias in a subject in need thereof by administering to the subject an effective amount our AN-233 optionally in combination or alternation with HU to increase the expression of HbF in the subject. β-thalassemias are a group of inherited blood disorders caused by a variety of mutational mechanisms that result in a reduction or absence of synthesis of β-globin and leading to accumulation of aggregates of unpaired, insoluble α-chains that cause ineffective erythropoiesis, accelerated red cell destruction, and severe anemia. Subjects with beta-thalassemia exhibit variable phenotypes ranging from severe anemia to clinically asymptomatic individuals. The genetic mutations present in β thalassemias are diverse, and can be caused by a number of different mutations. The mutations can involve a single base substitution or deletions or inserts within, near or upstream of the β globin gene. For example, mutations occur in the promoter regions preceding the beta-globin genes or cause production of abnormal splice variants.

Examples of thalassemias that can be treated with AN-233 optionally in combination or alternation with HU include thalassemia minor, thalassemia intermedia, and thalassemia major.

Thalassemia minor refers to thalassemia where only one of beta-globin alleles bears a mutation. Individuals typically suffer from microcytic anemia. Detection usually involves lower than normal MCV value (<80 fL) plus an increase in fraction of Hemoglobin A2 (>3.5%) and a decrease in fraction of Hemoglobin A (<97.5%). Genotypes can be β+/β or β−0/β.

Thalassemia intermedia refers to a thalassemia intermediate between the major and minor forms. Affected individuals can often manage a normal life but may need occasional transfusions, e.g., at times of illness or pregnancy, depending on the severity of their anemia. Genotypes can be β+/β+ or β−0/β.

Thalassemia major refers to a thalassemia where both beta-globin alleles have thalassemia mutations. This is a severe microcytic, hypochromic anemia. If left untreated, it causes anemia, splenomegaly, and severe bone deformities and typically leads to death before age 20. Treatment consists of periodic blood transfusion; splenectomy if splenomegaly is present, and treatment of transfusion-caused iron overload. Cure is possible by bone marrow transplantation. Cooley's anemia is named after Thomas Benton Cooley. Genotypes include β+/β−0 or β−0/β−0 or β+/β+.

3. Sickle Cell Related Disorders

Although carriers of sickle cell trait do not suffer from SCD, individuals with one copy of HbS and one copy of a gene that codes for another abnormal variant of hemoglobin, such as HbC or Hb beta-thalassemia, have a less severe form of the disease. For example, another specific defect in beta-globin causes another structural variant, hemoglobin C (HbC). Hemoglobin C (abbreviated as Hb C or HbC) is an abnormal hemoglobin in which substitution of a glutamic acid residue with a lysine residue at the 6^{th} position of the β-globin chain has occurred. A subject that is a double heterozygote for HbS and HbC (HbSC disease) is typically characterized by symptoms of moderate clinical severity.

Another common structural variant of beta-globin is hemoglobin E or hemoglobin E (HbE). HbE is an abnormal hemoglobin in which substitution of a glutamic acid residue with a lysine residue at the 26^{th} position of the β-globin chain has occurred. A subject that is a double heterozygote for HbS and HbE has HbS/HbE syndrome, which usually causes a phenotype similar to HbS/b+thalassemia, discussed below.

Some mutations in the beta-globin gene can cause other structural variations of hemoglobin or can cause a deficiency in the amount of β-globin being produced. These types of mutations are referred to as beta-thalassemia mutations.

The absence of beta-globin is referred to as beta-zero (β−0) thalassemia. A subject that is a double heterozygote for HbS and β−0 thalassemia (i.e., HbS/β−0 thalassemia) can suffer symptoms clinically indistinguishable from sickle cell anemia.

A reduced amount of beta-globin is referred to as β-plus (β+) thalassemia. A subject that is a double heterozygote for HbS and β+thalassemia (i.e., HbS/β+thalassemia) can have mild-to-moderate severity of clinical symptoms with variability among different ethnicities.

Rare combinations of HbS with other abnormal hemoglobins include HbD Los Angeles, G-Philadelphia, HbO Arab, and others.

Therefore, in some embodiments, the disclosed compositions and methods are used to treating a subject with an HbS/β−0 genotype, an HbS/β+ genotype, an HBSC genotype, an HbS/HbE genotype, an HbD Los Angeles genotype, a G-Philadelphia genotype, or an abHbO Arab genotype.

As discussed above, retinopathy due to SCD can also be treated by administering an effective amount of a AN-233, optionally in combination or alternation with HU in amounts effective to induce expression of HbF in retinal cells, for example in RPE cells. Sickle retinopathy occurs when the retinal blood vessels get occluded by sickle red blood cells and the retina becomes ischemic, angiogenic factors are made in retina. In sickle cell disease, this occurs mostly in the peripheral retina, which does not obscure vision at first. Eventually, the entire peripheral retina of the sickle cell patient becomes occluded and many neovascular formations occur. Administration of AN-233 optionally in combination with HU can reduce or inhibit the formation of occlusions in the peripheral retina of a sickle cell patient.

4. Non-Erythroid Cell Related Disorders

Although red blood cells are the primary producers of hemoglobin, reports indicate that other, non-hematopoietic cells, including, but not limited to, macrophage, retinal pigment cells, and alveolar epithelial cells such as alveolar type II (ATII) cells and Clara cells which are the primary producers of pulmonary surfactant, also synthesize hemoglobin (Newton, et al., J. Biol. Chem., 281(9)5668-5676 (2006), Tezel, et al., Invest. Ophthalmol. Vis. Sci., 50(4):1911-9 (2009), Liu, et al., Proc. Natl. Acad. Sci. USA, 96(12)6643-6647 (1999)). These findings are consistent with the conclusion that the expression of hemoglobin by non-erythroid cells at interfaces where oxygen-carbon dioxide diffusion occurs may be an adaptive mechanism to facilitate oxygen transport (Tezel, et al., Invest. Ophthalmol. Vis. Sci., 50(4):1911-9 (2009).

Therefore, in some embodiments, the AN-233 compositions disclosed herein are used to increase HbF expression in non-erythroid cells including, but not limited to, macrophage, retinal pigment cells, and alveolar epithelial cells such as alveolar type II (ATII) cells and Clara cells. In some embodiments, the compositions disclosed herein are used to increase HbF expression in non-erythroid cells at interfaces where oxygen-carbon dioxide diffusion occurs, including, but not limited to the eyes and lungs. In some embodiments, the compositions are used to induce, increase, or enhance hemoglobin synthesis retinal pigment cells in an effective amount to prevent, reduce, or alleviate one or more symptoms of age-related macular degeneration or diabetic retinopathy.

D. Symptoms of Sickle Cell Disease, Beta-Thalassemias, and Related Disorders

In some embodiments, the AN-233 compositions disclosed herein are administered to a subject in an effective amount to treatment one or more symptoms of sickle cell disease, a beta-thalassemia, or a related disorder.

Beta-thalassemia can include symptoms such as anemia, fatigue and weakness, pale skin or jaundice (yellowing of the skin), protruding abdomen with enlarged spleen and liver, dark urine, abnormal facial bones and poor growth, and poor appetite.

In subjects with sickle cell disease, or a related disorder, physiological changes in RBCs can result in a disease with the following signs: (1) hemolytic anemia; (2) vaso-occlusive crisis; and (3) multiple organ damage from microinfarcts, including heart, skeleton, spleen, and central nervous system.

Chronic Hemolytic Anemia

SCD is a form of hemolytic anemia, with red cell survival of around 10-20 days. Approximately one third of the hemolysis occurs intravascularly, releasing free hemoglobin (plasma free hemoglobin [PFH]) and arginase into plasma. PFH has been associated with endothelial injury including scavenging nitric oxide (NO), proinflammatory stress, and coagulopathy, resulting in vasomotor instability and proliferative vasculopathy. A hallmark of this proliferative vasculopathy is the development of pulmonary hypertension in adulthood.

Vaso-Occlusive Crisis

Vaso-occlusive crisis occurs when the circulation of blood vessels is obstructed by sickled red blood cells, causing ischemic injuries. The most common complaint is of pain, and recurrent episodes may cause irreversible organ damage. One of the most severe forms is the acute chest syndrome which occurs as a result of infarction of the lung parenchyma. Vaso-occlusive crisis can be accompanied by a pain crisis which can occur suddenly and last several hours to several days.

The pain can affect any body part. It often involves the abdomen, bones, joints, and soft tissue, and it may present as dactylitis (bilateral painful and swollen hands and/or feet in children), acute joint necrosis or avascular necrosis, or acute abdomen. With repeated episodes in the spleen, infarctions and autosplenectomy predisposing to life-threatening infection are usual. The liver also may infarct and progress to failure with time. Papillary necrosis is a common renal manifestation of vaso-occlusion, leading to isosthenuria (i.e, inability to concentrate urine).

Severe deep pain is present in the extremities, involving long bones. Abdominal pain can be severe, resembling acute abdomen; it may result from referred pain from other sites or intra-abdominal solid organ or soft tissue infarction. Reactive ileus leads to intestinal distention and pain.

Bone pain and abdominal pain may be present. The face also may be involved. Pain may be accompanied by fever, malaise, and leukocytosis.

Skeletal Manifestations

Skeletal manifestations include, but are not limited to, infarction of bone and bone marrow, compensatory bone marrow hyperplasia, secondary osteomyelitis, secondary growth defects, intravascular thrombosis, osteonecrosis (avascular necrosis/aseptic necrosis), degenerative bone and joint destruction, osteolysis (in acute infarction), Articular disintegration, myelosclerosis, periosteal reaction (unusual in the adult), Hvertebrae (steplike endplate depression also known as the Reynold sign or codfish vertebrae), Dystrophic medullary calcification, bone-within-bone appearance, decreased density of the skull, decreased thickness of outer table of skull due to widening of diploe, hair on-end striations of the calvaria, osteoporosis sometimes leading to biconcave vertebrae, coarsening of trabeculae in long and flat bones, and pathologic fractures, bone shortening (premature epiphyseal fusion), epiphyseal deformity with cupped metaphysis, peg-in-hole defect of distal femur, and decreased height of vertebrae (short stature and kyphoscoliosis).

Renal Manifestations

Renal manifestations include, but are not limited to, various functional abnormalities such as hematuria, proximal tubule dysfunction, impaired potassium excretion, and hyperkalemia; and gross anatomic alterations, for example, hypertrophied kidneys, with a characteristic smooth, capsular surface.

Splenic Manifestations

Splenic manifestations include, but are not limited to, enlargement, including rapid and/or painful enlargement known as splenic sequestration crisis, infarction, low pH and low oxygen tension in the sinusoids and splenic cords, functional impairment, autosplenectomy (fibrosis and shrinking of the spleen in advanced cases), immune deficiency and increased risk of sepsis.

Other Common Symptoms

Lower serum immunoglobulin M (IgM) levels, impaired opsonization, and sluggish alternative complement pathway activation, increase susceptibility to infection pneumonia, bronchitis, cholecystitis, pyelonephritis, cystitis, osteomyelitis, meningitis, and sepsis and other challenges from infectious agents including, but not limited to, Mycoplasma pneumoniae, Salmonella typhimurium, Staphylococcus aureus, and Escherichia coi; growth delays or maturation delays during puberty in adolescents, hand-foot syndrome, acute chest syndrome, stroke, hemiparesis, hemosiderin deposition in the myocardium, dilation of both ventricles and the left atrium, cholelithiasis, paraorbital facial infarction, retinal vascular changes, proliferative retinitis, loss of vision, leg ulcers, priapism, avascular necrosis, and pulmonary hypertension disorder or condition and/or preventing or eliminating said symptoms.

### III. Compositions and Formulations

One embodiment provides a composition containing an effective amount of AN-233 to increase expression of HbF in a subject in need thereof. The composition optionally includes an effective amount of HU to treat a hemaglobinopathy including SCD or a thalassemia. One embodiment provides a pharmaceutical composition containing an effective amount of AN-233 optionally in combination with HU to increase HbF expression in a subject in need thereof. In some embodiments the dosage is 1 mg/kg to about 50 mg/kg. In other embodiments the dosage is 0.1 g and 2.0 g per day. AN-233 optionally in combination or alternation with HU can be administered as part of a dosage regime. The dosage regime can include dose escalation.

A. Co-Administration

The AN-233 compositions disclosed herein can optionally include, or be co-administered with one or more additional therapeutic or active agents. Representative additional therapeutic agents include, but are not limited to L-glutamine oral powder, crizanlizumab, Voxelotor, fumaric esters, pain relieving drugs, and combinations thereof. Examples of suitable fumaric acid esters include, but are not limited to monoethyl fumarate (MEF), monomethyl fumarate (MMF), diethyl fumarate (DEF), and dimethyl fumarate (DMF).

Co-administration can include the simultaneous and/or sequential administration of the one or more additional active agents and AN-233, or pharmacologically active salt, derivative, or analogue thereof. The one or more additional active agents and AN-233, or pharmacologically active salt, derivative, or analogue thereof can be included in the same or different pharmaceutical formulation. The one or more additional active agents and AN-233, or pharmacologically active salt, derivative, or analogue thereof can achieve the same or different clinical benefit. An appropriate time course for sequential administration may be chosen by the physician, according to such factors as the nature of a patient's illness, and the patient's condition. In certain embodiments, sequential administration includes the co-administration of one or more additional active agents within a period of one week, 72 hours, 48 hours, 24 hours, or 12 hours.

The additional active agent can be chosen by the user based on the condition or disease to be treated. Example of additional active agents include, but are not limited to, vitamin supplements, nutritional supplements, anti-anxiety medication, anti-depression medication, anti-coagulants, clotting factors, anti-inflammatories, steroids such as corticosteroids, analgesic, etc.

In some embodiments, the compositions disclosed herein are co-administered in combination with one or more additional active agents for treatment of sickle cell disease, beta-thalassemia, or a related disorder. Such additional active agents may include, but are not limited to, folic acid, penicillin or another antibiotics, preferably a quinolone or macrolide, antivirals, anti-malarial prophylactics, and analgesics to control pain crises.

In some embodiments, the compositions are co-administered with one or more additional agents that increase expression of HbF, for example, hydroxyurea.

In some embodiments, the compositions are co-administered with one or more additional treatment protocols, for example, transfusion therapy, stem cell therapy, gene therapy, bone marrow transplants, dialysis or kidney transplant for kidney disease, gallbladder removal in people with gallstone disease, hip replacement for avascular necrosis of the hip, surgery for eye problems, and wound care for leg ulcers.

B. Effective Amounts

In some embodiments, the AN-233 compositions are administered in an amount effective to induce a pharmacological, physiological, or molecular effect compared to a control that is not administered the composition. In some embodiments, AN-233 optionally in combination with HU is administered to a subject in need thereof to increase expression of HbF in the subject. For example, HbF expression can be increased in an amount effective to compensate for, or reduce the effects of a mutation in the HBB gene. In some embodiments, the AN-233 composition is administered in an effective amount to reduce the sickling of red blood cells in a patient relative to a control.

In some embodiments, the AN-233 composition is provided in an effective amount to prevent, reduce or alleviate one or more symptoms of a disease or disorder to be treated. For example, the compositions disclosed herein can be administered to a subject in need thereof in an effective amount to reduce or alleviate one or more symptoms of sickle cell disease, a beta-thalassemia, or a sickle cell related disorder, including, but not limited to, the symptoms discussed above.

Suitable controls are known in the art and can be determined based on the disease to be treated. Suitable controls include, but are not limited to a subject, or subjects without sickle cell disease, a beta-thalassemia, or a sickle cell related disorder; or a condition or status of a subject with the disease or disorder prior to initiation of the treatment. For example, in some embodiments, treatment of a subject with an AN-233 composition improves one or more pharmacological, physiological, or molecular effects; reduces or alleviates one or more symptoms of the disease or disorder to be treated; or a combination thereof compared to a subject or subjects without the disease or disorder to be treated. In some embodiments, treatment of a subject with an AN-233 composition improves one or more pharmacological, physiological, or molecular effects; reduces or alleviates one or more symptoms of the disease or disorder to be treated; or a combination thereof in the subject compared to the same pharmacological, physiological, or molecular effects; or symptoms of the disease or disorder in the subject prior to administration of the AN-233 composition to the subject.

In some embodiments, the AN-233 composition is administered to a subject in need thereof in an effective amount to improve one or more pharmacological, physiological, or molecular effects, or to reduce or alleviate one or more symptoms of the disease or disorder with higher efficacy, lower toxicity, or a combination thereof compared to a subject treated with an different therapeutic agent such as hydroxyurea (HU).

C. Dosages and Dosage Regimes

For all of the disclosed AN-233 compositions, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 100 mg/kg of body weight daily are administered to mammals. Generally, for intravenous injection or infusion, dosage may be lower.

In some embodiments the compositions include AN-233. For AN-233, the therapeutically effective amount can range from about 1 mg/kg to about 50 mg/kg (e.g., from about 2.5 mg/kg to about 20 mg/kg or from about 2.5 mg/kg to about 15 mg/kg). Effective doses will also vary, as recognized by those skilled in the art, dependent on route of administration, excipient usage, and the possibility of co-usage with other therapeutic treatments including use of other therapeutic agents. For example, an effective dose of AN-233 to be administered to a subject, for example orally, can be from about 0.1 g to about 1 g or more than 1 g per day; from about 200 mg to about 800 mg per day; from about 240 mg to about 720 mg per day; from about 480 mg to about 720 mg per day; or about 720 mg per day. The daily dose can be administered in separate administrations of 2, 3, 4, or 6 equal doses.

In some embodiments AN-233 is formulated as a pharmaceutical composition or preparation. In some embodiments the composition is administered to the patient three times per day (TID). In some embodiments the pharmaceutical preparation is administered to the patient two times per day (BID). In some embodiments, the composition is administered at least one hour before or after food is consumed by the patient.

In some embodiments, the v composition is administered as part of a dosing regimen. For example, the patient can be administered a first dose of the AN-233 composition for a first dosing period; and a second dose of the AN-233 composition for a second dosing period, optionally followed by one or more additional doses for one or more additional dosing periods. The first dosing period can be less than one week, one week, or more than one week.

In some embodiments the dosage regime is a dose escalating dosage regime. The first dose can be a low dose. For example, in some embodiments, the composition includes AN-233, for example about 30 mg, can be the starting dose for a dose-escalation protocol. Dose escalation can be continued until a satisfactory biochemical or clinical response is reached. Next, the dosages can be maintained or steadily reduced to a maintenance dose. In some embodiments, the final dosage can be about 1-2 grams per day.

The current labeled dosing of hydroxyurea for sickle cell disease calls for the administration of an initial dose of 15 mg/kg/day in the form of a single dose, with monitoring of the patient's blood count every 2 weeks. If the blood counts are in an acceptable range, the dose may be increased by 5 mg/kg/day every 12 weeks until the MTD of 35 mg/kg/day is reached. Pharmaceutical compositions can contain 1 mg/kg to 50 mg/kg of AN-233 in combination with 1 mg/kg to 35 mg/kg of HU. The combination formulation can contain 5, 10, 15, 20, 25, 30, 35, 40, 45 or 50 mg/kg of HU.

D. Formulations

Pharmaceutical compositions including AN-233 are disclosed. The pharmaceutical compositions may be for administration by oral, parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in unit dosage forms appropriate for each route of administration.

Red blood cells, which are cells of erythroid lineage, are the primary producers of hemoglobin. Therefore, in a preferred embodiment AN-233 is administered to a subject in an effective amount to induce HbF expression in hematopoietic stems cells. In the early fetus, erythropoiesis takes place in the mesodermal cells of the yolk sac. By the third or fourth month, erythropoiesis moves to the spleen and liver. After seven months, erythropoiesis occurs primarily in the bone marrow, however, in certain disease states erythropoiesis can also occurs outside the bone marrow, within the spleen or liver, in adults. Therefore, in some embodiments, the compositions are administered in an effective amount to induce HbF expression in cells of erythroid lineage in the bone marrow (i.e., the red bone marrow), the liver, the spleen, or combinations thereof.

Preferably the composition induces HbF in cells synthesizing or committed to synthesize hemoglobin. For example, in preferred embodiments, AN-233 induces HbF in basophilic normoblast/early normoblast also commonly called erythroblast, polychromatophilic normoblast/intermediate normoblast, orthochromatic normoblast/late normoblast, or a combination thereof.

In a preferred embodiment, the composition is an oral formulation. Oral formulations of AN-233 can be absorbed by the small intestine where AN-233 can enter systemic circulation.

In some embodiments, the composition is administered locally, to the site in need of therapy. Although red blood cells are the primary producers of hemoglobin, reports indicate that other, non-hematopoietic cells, including macrophage, retinal pigment cells, and alveolar epithelial cells such as alveolar type II (ATII) cells and Clara cells which are the primary producers of pulmonary surfactant, also synthesize hemoglobin (Newton, et al., J. Biol. Chem., 281(9)5668-5676 (2006), Tezel, et al., Invest. Ophthalmol. Vis. Sci., 50(4):1911-9 (2009), Liu, et al., Proc. Natl. Acad. Sci. USA, 96(12)6643-6647 (1999)). These findings are consistent with the conclusion that the expression of hemoglobin by non-erythroid cells at interfaces where oxygen-carbon dioxide diffusion occurs may be an adaptive mechanism to facilitate oxygen transport.

Therefore, in some embodiments, the composition is administered locally to interfaces where oxygen-carbon dioxide diffusion occurs, including but not limited, to the eye or lungs.

In some embodiments, the composition is administered locally to the eye to treat a retinopathy, or another ocular manifestation associated with sickle cell disease, or a related disorder.

1. Formulations for Enteral Administration

In one embodiment the AN-233 compositions are formulated for oral delivery. Oral solid dosage forms are described generally in Remington's Pharmaceutical Sciences, 18^{th} Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc., or into liposomes. Such compositions may influence the physical state, stability, rate of in vivo release, and rate of in vivo clearance of the disclosed. See, e.g., Remington's Pharmaceutical Sciences, 18^{th} Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also, Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The AN-233 may be chemically modified so that oral delivery of the compound is efficacious. Generally, the chemical modification contemplated is the attachment of at least one moiety to the component molecule itself, where the moiety permits uptake into the blood stream from the stomach or intestine, or uptake directly into the intestinal mucosa. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. AN-233 can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects.

For oral formulations, the location of release may be the stomach, the small intestine (the duodenum, the jejunem, or the ileum), or the large intestine. Preferably, the release will avoid the deleterious effects of the stomach environment, either by protection of the agent (or derivative) or by release of the agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™ cellulose acetate phthalate (CAP), Eudragit L™, Eudragit S™, and Shellac™. These coatings may be used as mixed films.

2. Topical or Mucosal Delivery Formulations

In some embodiments that AN-233 compositions can be applied topically. The compositions can be delivered to the lungs while inhaling and traverses across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to, nebulizers, metered dose inhalers, and powder inhalers, all of which are familiar to those skilled in the art. Some specific examples of commercially available devices are the Ultravent™ nebulizer (Mallinckrodt Inc., St. Louis, Mo.); the Acorn II™ nebulizer (Marquest Medical Products, Englewood, Colo.); the Ventolin™ metered dose inhaler (Glaxo Inc., Research Triangle Park, N.C.); and the Spinhaler™ powder inhaler (Fisons Corp., Bedford, Mass.).

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations will require the inclusion of penetration enhancers.

3. Controlled Delivery Polymeric Matrices

Some embodiments provide controlled release polymeric devices made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of disclosed compounds, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release 5:13-22 (1987); Mathiowitz, et al., Reactive Polymers 6:275-283 (1987); and Mathiowitz, et al., J. Appl. Polymer Sci. 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### IV. Methods of Diagnosis

The methods of treatment disclosed herein can include a first step of selecting a subject for treatment. In some embodiments, the subject is selected for treatment when the subject exhibits one or more of the clinical symptoms of sickle cell disease, beta-thalassemia, or a related disorder such as those discussed above. In some embodiments, the subject is selected for treatment when the subject exhibits a genetic or biochemical indicator of sickle cell disease, beta-thalassemia, or a related disorder. For example, the subject can be selected for treatment based on identification of a genetic alteration, defect, or mutation in the beta-globin gene or an expression control sequence thereof, by biochemical or morphological alterations in hemoglobin or hemoglobin synthesizing cells, or combinations thereof.

In some embodiments, the subject is selected when a combination of clinical symptoms and genetic or biochemical alterations are identified. In some embodiments, the subject is selected based on one or more clinical symptoms, or one or more genetic or biochemical alterations. For example, subjects can be selected for treatment based on the identification of a genetic alteration, a biochemical or morphological alteration, or a combination thereof, before the subject exhibits clinical symptoms of sickle cell disease, beta-thalassemia, or a related disorder.

A. Identification of Genetic Alterations

In some embodiments, the subject is selected for treatment based on identification of one or more genetic alterations in one or more alleles of the human beta-globin gene or expression control sequence thereof. Genetic alterations indicative of sickle cell disease, beta-thalassemia, or related disorders include the exemplary mutations discussed above, or other mutations that lead to a reduction in the synthesis, structure, or function of human beta-globin protein.

Methods of selecting a subject having one or more genetic alterations in one or more alleles of the beta-globin gene or expression control sequences thereof include the steps of obtaining a biological sample containing nucleic acid from the subject and detecting the presence or absence one or more genetic alterations in one or more alleles of the beta-globin gene or expression control sequences thereof in the biological sample. Any biological sample that contains the DNA of the subject to be diagnosed can be employed, including tissue samples and blood samples, with nucleated blood cells being a particularly convenient source. The DNA may be isolated from the biological sample prior to testing the DNA for the presence or absence of the genetic alterations.

The detecting step can include determining whether the subject is heterozygous or homozygous for a genetic alteration. The step of detecting the presence or absence of the genetic alteration can include the step of detecting the presence or absence of the alteration in both chromosomes of the subject (i.e., detecting the presence or absence of one or two alleles containing the marker or functional polymorphism). More than one copy of a genetic alterations (i.e., subjects homozygous for the genetic marker) can indicate a greater risk of developing sickle cell disease, beta-thalassemia, or related disorder. In some embodiments, the subject is heterozygous for two or more genetic alterations in the beta-globin gene (also referred to herein as double heterozygotes, triple heterozygotes, etc.). One copy of two or more genetic alterations in the beta-globin gene can indicate a greater risk of developing sickle cell disease, beta-thalassemia, or related disorder.

The process of determining the genetic sequence of human beta-globin gene is referred to as genotyping. In some embodiments, the human beta-globin gene is sequenced. Methods for amplifying DNA fragments and sequencing them are well known in the art. For example, automated sequencing procedures that can be utilized to sequence the beta-globin gene, include, but not limited to, sequencing by mass spectrometry single-molecule real-time sequencing (Pacific Bio), ion semiconductor (ion torrent sequencing), pyrosequencing (454), sequencing by synthesis (Illumina), sequencing by ligation (SOLiD sequencing), chain termination (Sanger sequencing).

In some embodiments, the genotype of the subject is determined by identifying the presence of one or more single nucleotide polymorphisms (SNP) associated with sickle cell disease, beta-thalassemia, or a related disorder. Methods for SNP genotyping are generally known in the art (Chen et al., Pharmacogenomics J., 3(2):77-96 (2003); Kwok, et al., Curr. Issues Mol. Biol., 5(2):43-60 (2003); Shi, Am. J Pharmacogenomics, 2(3):197-205 (2002); and Kwok, Annu. Rev. Genomics Hum. Genet., 2:235-58 (2001)).

SNP genotyping can include the steps of collecting a biological sample from a subject (e.g., sample of tissues, cells, fluids, secretions, etc.), isolating genomic DNA from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

The neighboring sequence can be used to design SNP detection reagents such as oligonucleotide probes and primers. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Other suitable methods for detecting polymorphisms include methods in which protection from cleavage agents is used to detect mismatched bases in RNA/RNA or RNA/DNA duplexes (Myers et al., Science, 230:1242 (1985); Cotton, et al., PNAS, 85:4397 (1988); and Saleeba, et al., Meth. Enzymol., 217:286-295 (1992)), comparison of the electrophoretic mobility of variant and wild type nucleic acid molecules (Orita et al., PNAS, 86:2766 (1989); Cotton, et al, Mutat. Res., 285:125-144 (1993); and Hayashi, et al., Genet. Anal. Tech. Appl., 9:73-79 (1992)), and assaying the movement of polymorphic or wild-type fragments in polyacrylamide gels containing a gradient of denaturant using denaturing gradient gel electrophoresis (DGGE) (Myers et al., Nature, 313:495 (1985)). Sequence variations at specific locations can also be assessed by nuclease protection assays such as Rnase and Sl protection or chemical cleavage methods.

Another method for genotyping SNPs is the use of two oligonucleotide probes in an oligonucleotide ligation assay (OLA) (U.S. Pat. No. 4,988,617). In this method, one probe hybridizes to a segment of a target nucleic acid with its 3′-most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′-most nucleotide of the first probe with the SNP site. If there is a mismatch, ligation would not occur. After the reaction, the ligated probes are separated from the target nucleic acid molecule, and detected as indicators of the presence of a SNP.

Other methods that can be used to genotype the SNPs include single-strand conformational polymorphism (SSCP), and denaturing gradient gel electrophoresis (DGGE). SSCP identifies base differences by alteration in electrophoretic migration of single stranded PCR products. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel.

Sequence-specific ribozymes (U.S. Pat. No. 5,498,531) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis.

B. Identification of Biochemical and Morphological Alterations

In some embodiments, subjects are selected for treatment based on identification of biochemical or morphological alterations or abnormalities in hemoglobin, or hemoglobin synthesizing cells such as hematopoietic stem cells, erythrocyte progenitor cells, erythrocytes, macrophage, retinal pigment epithelial cells, alveolar type II (ATII) cells, and others. The methods typically include identifying one or more biochemical or morphological alterations that is associated with a genetic alteration in the human beta-globin gene, or otherwise diagnostic of sickle cell disease, a beta-thalassemia, or a related disorder. Methods of diagnosing sickle cell disease, beta-thalassemia, or a related disorder according to biochemical or morphological alterations in the hemoglobin or hemoglobin synthesizing cells are known in the art, and include but are not limited to, analysis of erythrocyte morphology, osmotic fragility, hemoglobin composition, globin synthetsis rates, and red blood cell indices (Rowley, American Journal of Hematology, 1(1):129-137, (1976)).

In some embodiments, the method includes testing a subject's blood for HbS, and selecting the subject for treatment if HbS is present. Methods for testing a subject's blood for the presence of HbS include solubility tests (e.g., SICKLEDEX) and sickling test. The SICKLEDEX test operates on the principle that Hb-S tends to form tactoids or liquid crystals within the erythrocytes under conditions of low oxygen tension resulting in the characteristic “sickle shape” distortion of the red cell. A reducing agent (i.e., dithionite) is mixed with whole blood and buffer. If Hb-S is present, it becomes insoluble and forms a cloudy suspension. Other hemoglobins are more soluble and will form a transparent solution. A sickling test can be used to determine if a red blood cell changes into a sickle shape after a blood sample is mixed with a reducing agent and identifying morphological changes to shape of red blood cells (i.e., “sickling”) by microscopy.

Other suitable tests include, hemoglobin electrophoresis, which employs gel electrophoretic techniques to separate out the various types of hemoglobin from a blood sample obtained from the subject. The test can detect abnormal levels of HbS, as well as other abnormal hemoglobins, such as hemoglobin C. It can also be used to determine whether there is a deficiency of any normal form of hemoglobin, as in various thalassemias. Alternatives to electrophoretic techniques include isoelectric focusing and chromatographic techniques.

Other tests that can be used to select a subject for treatment with the compositions and methods disclosed herein include tests typically employed as part of a hemoglobinopathy screen, for example, a complete blood count (CBC) or iron study (ferritin). For example, a blood count can be used to detect anemia, and a blood smear and be used to identify sickled cells.

### EXAMPLES

### Example 1: AN-233 Induces γ-Globin Transcription and HbF Expression in K562 Cells

Materials and Methods

Synthesis of AN-233 Prodrug

The conjugate prodrug AN-233 was synthesized as a precursor compound of BA attached to ALA with a protective BOC (tert-butylox-ycarbonyl) group on the N terminal by Drs. Rephaeli and Nudelman (Berkovitch, G., et al., J. Med. Chem., 51:7356-7369 (2008)). Purified AN-233 was obtained by the removal of the BOC protective group under acidic conditions to yield the acyloxyethyl esters of ALA and BA. Synthesized AN-233 was made available with >95% purity in two lots. The first lot was reconstituted in 10% ethanol (vehicle control) while the second lot was reconstituted in water for in vivo studies to avoid potential toxicities in mice.

Tissue Culture and Reagents

Initial studies were conducted in K562 cells to determine the ability of AN-233 to induce 7-globin transcription and HbF expression. K562 cells display characteristics of erythroid cells including expression of the F, 7 and a globin genes and these cells are useful for initial drug screening and discovery of potential HbF inducers.

K562 cells were treated with AN-233 for 48 h and globin gene transcription was analyzed by RT-qPCR. K562 cells were treated with AN-233 for 48 h and globin gene transcription was analyzed by RT-qPCR.

K562 cells were cultured in Iscove's Modified Dulbecco medium (IMDM) with 10% fetal bovine serum, penicillin (100 U/mL) and streptomycin (0.1 mg/mL). Drug inductions for K562 cells were conducted for 48 h and cell viability evaluated with 0.4% Trypan blue exclusion. Cell counts were performed a dual chamber apparatus and the percentage viability obtained using an Automated Cell Counter (Bio-Rad).

For primary cultures, erythroid precursors were generated from peripheral blood mononuclear cells isolated from discard blood of sickle patients under an IRB exempt protocol. These cells were cultured in a two-phase liquid culture system previously published (Li, B., et al., Haematologica 103:e384-e387 (2018)).

During phase 1, cells were grown in Iscove's Dulecco Media with 15% fetal bovine serum, 15% human AB serum, 10 ng/mL interleukin-3, 50 ng/mL stem cell factor and 2 IU/mL of erythropoietin (Peprotech, Rocky Hill N.J.). Phase 2 of culture initiated on day 7 with a similar medium without stem cell factor and interleukin-3. On day 8, erythroid precursors were treated with AN-233 (0.125 mM and 0.25 mM), ethanol (EtOH; 0.0008% and 0.016%) and the positive control HU (100 μM) for 48 h and harvested for the various analyses.

Statistical Analysis

For tissue culture studies, data for at least 3-5 independent experiments performed in triplicate were reported as the mean±standard error of the mean (SEM). The Student's t-test was performed to determine significance and p<0.05 was considered statistically significant. For β-YAC studies, untreated (water), HU and AN-233 treated mice were analyzed by paired t-tests to compare week 0 (baseline) to week 2 and week 4. Data were normalized based on 100×[activity (therapeutic)-mean activity (negative control)]/[Activity(positive control)-mean activity (negative control)]. Finally, changes across treatment groups were compared using ANOVA with post-hoc Tukey HSD test for pairwise comparison at week 2 and week 4.

Reverse Transcription-Quantitative PCR (RT-qPCR) Analysis

Total RNA was extracted from cells using Trizol (Ambion, Carlsbad Calif.) and analyzed by RT-qPCR as previously published by our group [12]. Gene-specific primers were used to quantify mRNA levels for γ-globin, β-globin and internal control glyceraldehyde-3-phosphate de-hydrogenase (GAPDH). All mRNA levels were normalized to GAPDH before analysis.

Western Blot Analysis

Western blot analysis was performed using whole cell lysates generated with RIPA buffer (ThermoScientific, Rockford, Ill.) supplemented with proteinase and phosphatase inhibitor cocktails. For histone acetylation studies, nuclear lysates were prepared by suspending cells in buffer containing 20 mM HEPES, pH 7.9, 50 mM KCl, 420 mM NaCl, 0.1 mM EDTA, 1 mM DTT, 10% glycerol and protease inhibitor mixture for 30 min, followed by centrifugation. Antibodies against HbF (51-7), HbA (37-8), and Tata binding protein (TBP; N-12) were purchased from Santa Cruz Biotechnology (Dallas Tex.); antibodies against β-actin (A5316) and rabbit IgG (I8140) were purchased from Sigma (St. Louis Mo.). Acetylated histone H3 (AcH3; 06-599) and AcH4 (06-866) antibodies were purchased from Millipore (Burlington, Mass.).

Flow Cytometry Analysis

To measure percent HbF positive cells (F-cells), K562 cells and erythroid precursors were fixed with 1% formaldehyde, permeabilized with ice-cold acetone:methanol (4:1 ratio) and stained with fluorescein isothiocyanate (FITC) anti-HbF antibody (ab19365, Abcam Cambridge Mass.) and isotype control IgG antibody (MBS524511, MyBioSource, San Diego Calif.) was used to detect non-specific staining. The F-cells levels and HbF protein levels measured by mean fluorescence intensity (MFI) were analyzed on an LSR-II flow cytometer (BD Biosciences, San Jose Calif.) and FlowJo analysis to generate quantitative data.

Results

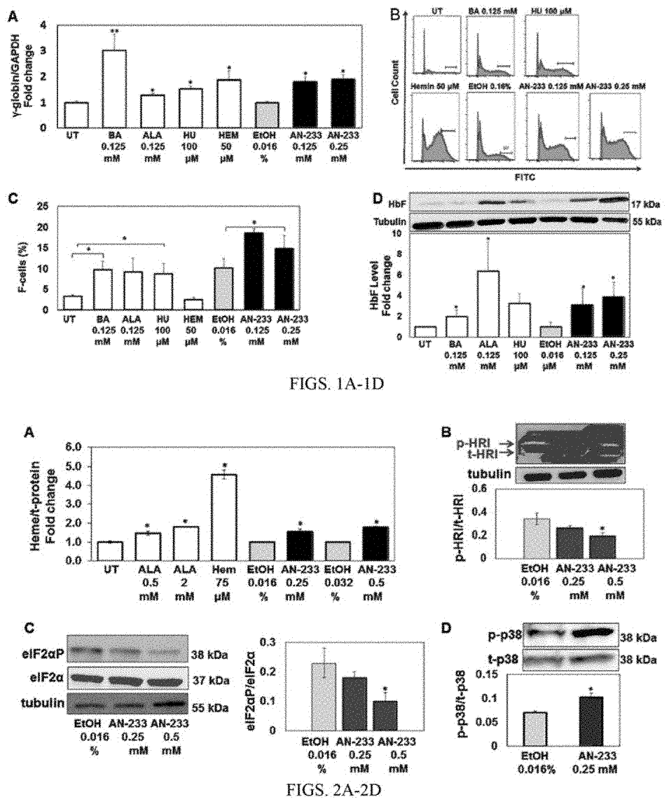

A significant increase in 7-globin mRNA levels of 1.8-fold and 2.0-fold by AN-233 at 0.125 and 0.25 mM respectively (FIG. 1A) compared to 1.7-fold induction by HU were observed. Control studies with BA and ALA alone directly added in culture at 0.125 mM, increased 7-globin mRNA 3-fold and 1.5 fold respectively. The next set of studies determined the effects of AN-233 on HbF protein expression by flow cytometry. Similar to mRNA levels, treatment with AN-233 (0.125 and 0.25 mM) increased the F-cells to a maximum of 19% compared to 10% in EtOH treated controls (FIGS. 1B and 1C). To substantiate HbF protein levels, Western blot was performed confirming a dose-dependent 3 to 4-fold increase in HbF (p<0.05) by AN-233 (FIG. 1D). These levels compare to a 2-fold and 6-fold increase in HbF by BA and ALA respectively (p<0.05).

### Example II: AN-233 Stimulates Heme Biosynthesis and Regulates Cellular Protein Targets

Materials and Methods

Heme Quantitation Assay

The total cellular heme content was determined using the QuantiChrom™ Heme Assay Kit (DIHM-250, BioAssay Systems, Hayward, Calif.) per the manufacturer's instructions. Briefly, 25 μL of cellular lysate was mixed with 100 μL of detection reagent. The mixture was incubated at room temperature for 5 min followed by measuring the absorbance at 400 nm on a microplate reader. The total heme concentration was calculated based on a formula provided by the manufacturer: Heme concentration=(OD^{sample}−OD^{blank})/(OD^{calibrator}−OD^{blank})×125×dilution factor. This value was normalized by total protein in each sample.

Results

Since the biosynthesis of heme prosthetic group requires ALA as a precursor and AN-233 is hydrolyzed to BA and ALA, it was next determined whether heme levels are altered after treatments of K562 cells. Using a colorimetric quantitative assay, a 1.5-fold and 1.8-fold increase (p<0.05) in intracellular heme was observed after 0.25 mM and 0.5 mM AN-233 treatment respectively (FIG. 2A). As would be expected, ALA (0.5 and 2.0 mM) and hemin (75 μM) increased heme levels in control experiments. Cellular heme is known to directly modulate the activity of Heme-regulated inhibitor (HRI) kinase, which under iron deficient states is activated to mediate eIF2αP (eukaryotic translation initiation factor 2α phosphorylation) and inhibition of protein synthesis. Therefore, K562 cells were treated with AN-233 and phosphorylated and total HRI and eIF2αP levels were measured. A dose-dependent maximal 52% decrease in HRI and 50% decrease in eIF2αP levels by AN-233 (FIGS. 2B and C) was observed.

The second active metabolite of AN-233 hydrolysis is BA. It was previously reported that HbF induction by BA occurs through p38 MAPK activation. Western blot analysis showed that 0.25 mM AN-233 induced a 1.4-fold increase in phosphorylated p38 MAPK (FIG. 2D). By contrast, pretreatment with the p38 MAPK inhibitor SB203580 (10 μM) followed by 0.25 mM AN-233, decreased F-cell levels by 30% suggesting p38 signaling is partially involved in mechanisms of HbF induction by AN-233.

### Example III: AN-233 Induces HbF Synthesis in Sickle Erythroid Precursors and Inhibits Sickling

Materials and Methods

Sickling Assay

In vitro sickling studies were conducted as previously published by our group (Zhu, X., et al., Blood, 131:558-562 (2018)). Briefly, after drug inductions of sickle erythroid precursors for 48 h, cells were incubated in 2% oxygen overnight and then fixed with 2% formaldehyde. Erythroid morphology was evaluated microscopically and the number of sickled cells per high power field manually counted for 1000 cells, per triplicates per condition.

Results

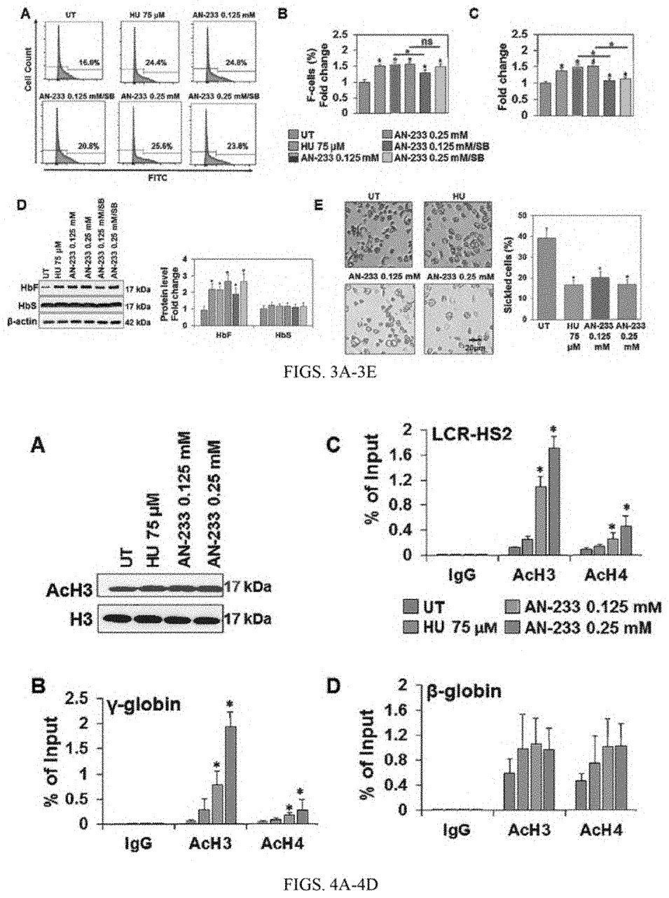

While K562 cells serve as an initial screening model system for HbF inducers, the findings were explored in physiologically relevant cells. Thus, sickle erythroid precursors were generated from peripheral blood mononuclear cells of SCD patients using a 2-phase liquid culture system. After AN-233 (0.125 and 0.25 mM) treatment, a maximal 2-fold increase was observed in F-cells from 16.31% to 32.5% (FIGS. 3A and 3B). Similarly, HbF measured by MFI increased 1.5-fold, levels comparable to HU treated cells (FIG. 3C). Pretreatment with SB203580 reduced F-cells and MFI levels at both AN-233 concentrations. To substantiate these findings in sickle precursors, Western blot confirmed the ability of 0.25 mM AN-233 to increase HbF protein by 2.6-fold (p<0.05) without changing HbS expression (FIG. 3D), which was inhibited by SB203580 treatment.

While HbF induction is a good indicator of drug efficacy, it is also desirable to achieve an anti-sickling effect under hypoxic conditions. Therefore, sickle erythroid precursors were incubated in 2% oxygen overnight, fixed with formaldehyde and examined by light microscopy. As shown in FIG. 3E AN-233 reduced the percentage of sickled erythroid precursors up to 56% (p<0.05) similar to HU, supporting an anti-sickling effects of the prodrug. Furthermore, pretreatment with SB203580 produced higher sickled erythroid progenitor levels. These findings support the ability of the AN-233 to induce HbF in sickle erythroid cells.

### Example IV: AN-233 Increased Histone Acetylation in β-Globin Locus

Materials and Methods

Chromatin Immunoprecipitation (ChIP) Assay

ChIP assay was performed as previously published by our group [22] with immunoprecipitations for AcH3, AcH4 and TBP antibodies (Santa Cruz Biotechnology) and rabbit IgG as a control. Primers used to quantify in vivo chromatin modifications are as follows: locus control region DNase I hypersensitive site 2 (LCR-HS2) forward CCTTCTGGCTCAAGCACAGC (SEQ ID NO:1) and reverse ATAGGAGTCATCACTCTAGGC (SEQ ID NO:2), γ-globin promoter (forward CTGAAACGGTCCCTGGCTA (SEQ ID NO:3), reverse CTGTGAAAT GACCCATGGCG (SEQ ID NO:4)), and β-globin promoter (forward TGGAGCCACACCCTAGGGTTGGC (SEQ ID NO:5), reverse CTTGTAACCTTGATACCAACCTG (SEQ ID NO:6)).

Results

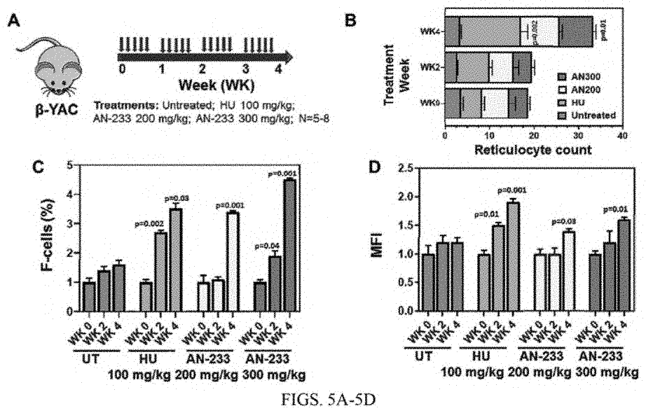

Previous work demonstrated the ability of BA to induce HbF via inhibition of histone deacetylases. Therefore, AcH3 and AcH4 levels were determined in nuclear lysates of sickle erythroid cells after AN-233 treatment, where increased global AcH3 levels were observed (FIG. 4A). It was next investigated whether histone acetylation levels are enhanced in the β-globin locus as part of mechanisms of γ-globin gene activation. To answer this question, ChIP assay demonstrated that AN-233 mediated a dose-dependent 12-fold and 30-fold enrichment for AcH3 in the γ-globin promoter compared to IgG control studies 4B); likewise, AcH4 levels were increased. Similar effects were observed in the LCR-HS2 region where AN-233 mediated 12.5-fold and 5-fold increase in AcH3 and AcH4 respectively (FIG. 4C). By contrast, there were no significant changes in histone acetylation at the β-globin gene promoter (FIG. 4D).

### Example V: AN-233 Induces HbF Expression in β-YAC Transgenic Mice

Materials and Methods

β-YAC Transgenic Mouse Treatment Protocol

The β-YAC is a transgenic mouse model containing the full-length 81 kb human β-globin gene locus including the LCR and surrounding region. The five functional human globin genes 5′-ε-Gγ-Aγ-8-β-3′ are present and undergo normal developmental regulation with the γ-globin gene silenced shortly after birth. 3-YAC mice (5-6 months old) were administered AN-233 suspended in water (200 or 300 mg/kg) 5 days/week for 4 weeks by intraperitoneal injection; five mice per group with 3 males and 2 females were treated. Hydroxyurea (100 mg/kg) was included as a positive control. Blood was collected by tail bleed at week 0, 2 and 4 and analyzed for automated complete blood counts with differential using a Micros 60 machine (HORIBA Medical/ABX Diagnostics). The level of F-cells and MFI were performed by flow cytometry. For reticulocyte counts, whole blood was stained with acridine orange and flow cytometry performed on an LSR-II flow cytometer (BD Biosciences).

Results

Many have shown drug-mediated HbF induction in tissue culture systems, these findings do not always translate in vivo. Therefore, the final preclinical studies evaluated the potential of AN-233 to induce HbF using -YAC transgenic mice, in which y-globin to -globin switching occurs during development. HbF induction by a-amino butyric acid in this model was previously demonstrated. Mice 5-6 months old were administered AN-233 dissolved in water (200 mg/kg and 300 mg/kg) or HU (100 mg/kg) by intraperitoneal injections, 5 days per week for 4 weeks with five mice per treatment group; an untreated water control group was also analyzed (FIG. 5A). At week 0, 2 and 4, mice were weighed and blood samples collected by tail bleed for automated complete blood counts and reticulocyte percent, percentage of F-cells and MFI by flow cytometry. Over 4 weeks of treatment, no drug toxicity occurred and normal body weights were maintained for all groups (data not shown). Untreated control mice had no significant change in blood counts over the 4-week treatment period, however, HU decreased total Hb, hematocrit, and white blood cell and platelet counts. Treatment with AN-233 at both doses produced a mild decrease in Hb and hematocrit, but the platelet count remained normal. To gain insights into the effects of AN-233 on erythropoiesis reticulocyte count was measured by acridine orange staining and flow cytometry. As shown in FIG. 5B, treatment with 300 mg/kg AN-233 increased reticulocytes 1.8-fold (p=0.01) at week 4, compared to a 1.9-fold increase for HU (p=0.002) suggesting AN-233 stimulated erythropoiesis.

The ability of AN-233 to induce HbF expression in vivo was analyzed. As shown in FIG. 5C, the F-cells increased 3.4-fold (from 15.46% to 52.5%) in mice treated with 200 mg/kg AN-233, while 300 mg/kg increased F-cells 4.5-fold (from 13.5% to 60.6%). The levels of HbF measured by MFI increased 1.4-fold (from 588 to 824 units) and 1.7-fold (from 447 to 760 units) respectively at the two AN-233 doses (FIG. 5D). With five mice per treatment group, ANOVA was performed, which showed significant difference between untreated (water) control and all other treatment groups for Hb (p=0.0359), reticulocytes (p=0.0003), F-cells (p=0.0109) and MFI (p=0.0369) by week 4. These findings support the ability of AN-233 to induce HbF in vivo in 3-YAC transgenic mice.

### DISCUSSION

Over the last three decades, numerous pharmacologic agents have been tested and shown to display HbF inducing properties in vitro, but few have translated into clinical efficacy. However, HbF induction by small molecules is an important therapeutic approach for treatment of the β-hemoglobinopathies and continues to be an intense area of investigation. Agents such as 5-azacytidine, decitabine, arginine butyrate, and short chain fatty acid derivatives were shown to induce HbF in clinical trials. These drugs act by diverse mechanisms including inhibition of DNA methyl transferases and histone deacetylases, enhanced DNA binding of transcription factors and cell signaling activation. Recent studies of combined oral treatment with decitabine and tetrahydrouridine showed HbF induction in a Phase 1 clinical trial. Thus, development of additional safe and effective oral agents that induce HbF without bone marrow toxicity when combined with HU, offer the potential for improved outcomes in β-hemoglobinopathies.

K562 cells were used to provide in vitro evidence of HbF induction by the prodrug conjugate AN-233 composed of BA and ALA. Increased γ-globin transcription at the mRNA level and HbF protein synthesis were observed after AN-233 treatment. While the findings in K562 cells support efficacy, these cells arguably possess inherent features that make them less likely to recapitulate findings in erythroid cells. Therefore, drug induction studies were performed in human primary sickle erythroid precursors undergoing terminal differentiation to determine the ability of AN-233 to induce HbF under oxidative stress conditions.

Treatment with AN-233 significantly increased γ-globin mRNA and HbF levels in sickle erythroid cells. Interestingly, evaluation of changes in HbS protein revealed that the prodrug did not induce synthesis of adult globin chains. These effects of AN-233 are clinically desirable since drugs that either have no effect or decrease HbS levels would produce an anti-sickling result. Indeed, under hypoxic conditions a lower number of sickle precursors were observed after AN-233 treatment confirming an anti-sickling effect similar to that produced by HU.

After intracellular hydrolysis of AN-233 by esterase enzymes, two active metabolites BA and ALA are released. Therefore, to verify activity of these agents, mechanisms of HbF induction through pathways regulated by both compounds were tested. Butyric acid is a pan-histone deacetylase inhibitor, which mediates histones acetylation causing epigenetic changes in chromatin structure allowing accessibility of DNA binding proteins to activate gene transcription. By ChIP assay, it was confirmed that histone H3 and H4 acetylation levels were increased in the γ-globin gene promoter and LCR-HS2; by contrast, no significant change of histone acetylation occurred in the β-globin gene. A second mechanism by which BA induces HbF expression is through p38 MAPK phosphorylation to stimulate cell signaling and activation of CREB1 to achieve γ-globin gene transcription. To support this mechanism of AN-233, γ-globin gene silencing and a decrease in anti-sickling effects in primary erythroid precursors was observed when the p38 MAPK inhibitor SB203580 was added.

The second active metabolite of AN-233 hydrolysis is ALA, a known precursor of heme biosynthesis involving eight enzymes in the mitochondrion and cytoplasm of cells. Among them, δ-aminolevulinate synthase catalyzes the first and rate limiting reaction to produce ALA. The addition of exogenous ALA accelerates heme production and enhances globin mRNA translation and Hb synthesis in cell culture systems. To gain insights into heme-related mechanisms of HbF activation by AN-233, the HRI/eIF2αP signaling axis, normally regulated by heme levels in vivo was investigated. A significant increase in heme levels by AN-233 and simultaneous silencing of the protein kinases HRI and eIF2αP, which normally block global protein synthesis was observed. Recent studies from Blobel and colleagues showed HRI depletion markedly increased HbF production and reduced sickling in human primary erythroid cells. Furthermore, diminished expression of the major γ-globin repressor BCL11A accounted in part for the effects of HRI depletion.

Studies have shown that activation of the eIF2α stress pathway mediates HbF induction through post-transcriptional mechanisms. For example, salubrinal activates eIF2α signaling to enhance HbF production in primary human erythroid cells. Salubrinal selectively increased the number of actively translating ribosomes on γ-globin mRNA. Translational regulation of hemoglobin synthesis is mediated by HRI, which is an intracellular heme sensor that coordinates heme and globin synthesis during erythropoiesis. In iron deficient states, HRI is activated and inhibits synthesis of globin chains and heme biosynthetic enzymes. The HRI-eIF2αP-ATF4 stress signaling pathway is important for regulating excess globin synthesis during erythropoiesis, and for adaptation to oxidative stress. Modulation of this signaling pathway with small chemicals may provide a novel therapy for β-hemoglobinopathies.

To translate novel HbF inducers into clinical trials requires evidence of efficacy in preclinical animal models. The in vivo safety and efficacy of oral AN-233 was previously explored in an anemic C57BL mouse model; mice were treated for 4 weeks with up to 400 mg/kg without toxicity. In fact, hemoglobin levels improved and tissue harvested 4 to 6 h after one oral dose of AN-233 confirmed histone hyper-acetylation in spleen tissue. We treated β-YAC mice, 5 days per week for 4 weeks, without observing anti-proliferative effects of AN-233 on erythropoiesis. The mild increase in reticulocyte counts and decrease in hemoglobin suggest mild hemolysis, which requires additional studies.

The β-YAC mouse model has been used to test different agents for their capacity to induce HbF in vivo. We demonstrated the ability of α-amino butyric acid to activate γ-globin transcription when combined with 5-azacytidine. Subsequently, we tested the histone deacetylase inhibitor, Scriptaid, which activated γ-globin without affecting β-globin gene transcription. Others agents analyzed in β-YAC mice that induce γ-globin include tranylcypromine (LSD1 inhibitor), sodium dimethyl butyrate (histone deacetylase inhibitor) and benserazide (DOPA decarboxylase inhibitor) which support potential in vivo efficacy. Subsequent human trials with sodium dimethylbutyrate (HQK-1001) demonstrated HbF induction in 70% of patients with β-thalassemia, but was less effective in SCD. Even though 3-YAC mice undergo hemoglobin switching and serve as an excellent pre-clinical model for drug screening, limitation of the model included the lack of anemia and oxidative stress present in SCD and β-thalassemia. SCD mice or baboons provide additional animal models to test pharmacological agents for their potential to induce HbF.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A method for treating a hemoglobinopathy, a sickle cell-related disorder, or a beta thalassemia in a subject in need thereof, comprising administering to the subject a therapeutically effective amount of 1-(butyryloxy)ethyl-5-amino-4-oxopentanoate (AN-233).

2. The method of claim 1, which is a method for treating a hemoglobinopathy.

3. The method of claim 2, wherein the hemoglobinopathy is a sickle cell disorder.

4. The method of claim 3, wherein the sickle cell disorder is sickle cell anemia.

5. The method of claim 1, which is a method for treating a beta thalassemia.

6. The method of claim 1, which is a method for treating a sickle cell-related disorder.

7. The method of claim 6, wherein the sickle cell-related disorder is a retinopathy.

8. The method of claim 1, wherein the method further comprises administering hydroxyurea to the subject.

9. The method of claim 8, wherein the subject is unresponsive to treatment with hydroxyurea alone.

10. The method of claim 9, wherein the subject expresses lower levels of OCTN1 than patients who respond to hydroxyurea.

11. The method of claim 1, wherein the administering of the 1-(butyryloxy)ethyl-5-amino-4-oxopentanoate (AN-233) is orally.

12. The method of claim 7, wherein the administering of the 1-(butyryloxy)ethyl-5-amino-4-oxopentanoate (AN-233) is locally to the eye.

13. A pharmaceutical composition comprising an effective amount of AN-233 to increase HbF expression in a subject in need thereof.

14. The composition of claim 13, further comprising hydroxyurea.
